(19) United States
(12) Patent Application Publication
(10) Pub. No.: US 2026/0174942 A1
(43) Pub. Date: Jun. 25, 2026

(54) SORBENT DEVICE FOR REGENERATION OF DIALYSATE

(71) Applicant: NEXTKIDNEY SA, Lausanne (CH)

(72) Inventors: Christian BLUCHEL, Lausanne (CH); Sjaak JONG, Lausanne (CH); Paiboon KHAOPAIBUL, Lausanne (CH); Zi Jun KWOK, Lausanne (CH)

(21) Appl. No.: 19/125,067

(22) PCT Filed: Nov. 2, 2023
(86) PCT No.: PCT/IB2023/061036

§ 371 Date: Apr. 28, 2025

(30) Foreign Application Priority Data
Nov. 2, 2022 (EP) 22205023.9
Nov. 2, 2022 (EP) 22205027.0
Nov. 2, 2022 (EP) 22205030.4
Nov. 2, 2022 (EP) 22205036.1
Nov. 2, 2022 (EP) 22205040.3
Nov. 2, 2022 (EP) 22205043.7
Oct. 31, 2023 (WO) PCT/IB2023/060957

(51) Int. Cl. A61M 1/16; A61M 1/36; B01J 20/02; B01J 20/06; B01J 20/20

(52) U.S. Cl. CPC A61M 1/1672; A61M 1/16; A61M 1/3627; A61M 1/3679; B01J 20/0244; B01J 20/06; B01J 20/20

(57) ABSTRACT

The present disclosure relates to a system using a sorbent device or to a sorbent device configured for a dialysis treatment such as an extracorporeal blood treatment or a peritoneal dialysis treatment.

### TECHNICAL FIELD

The present disclosure relates to a system using a sorbent device or to a sorbent device configured for a dialysis treatment such as an extracorporeal blood treatment or a peritoneal dialysis treatment.

### BACKGROUND

Dialysis treatment is typically used to extract undesirable matter or molecules from the patient's blood and/or add desirable matter or molecules to the blood. Such treatment is used with patients unable to effectively remove matter from their blood, such as when a patient has suffered temporary or permanent kidney failure.

The extracorporeal blood treatment is accomplished by removing the blood from the patient, introducing the blood into a filtration unit (for example a dialyzer) where the blood is allowed to flow past a semipermeable membrane. The semipermeable membrane selectively allows matter in the blood to cross the membrane from a primary chamber into a secondary chamber and also selectively allows matter in the secondary chamber to cross the membrane into the blood in the primary chamber, depending on the type of treatment (ultrafiltration (UF) treatment, hemofiltration (HF) treatment, hemodialysis (HD) treatment, hemodiafiltration (HDF) treatment, . . . ).

The peritoneal dialysis treatment is accomplished by filling the patient's peritoneum with a dialysate solution and after a dwell stage, draining the dialysate solution from the patient. Peritoneal dialysis treatment and extracorporeal blood treatment (such as hemodialysis) are different treatments, but the concept remains similar: on one side is the patient's blood and on the other the dialysate. The membrane that separates the blood side from the dialysate side is either the peritoneal membrane or the dialyzer membrane.

The dialysis treatment is widely carried up out in medical centers, where caregivers operate the dialysis systems and ensure a safe treatment. But more and more treatments are performing at home and the patient is not always accompanied by a caregiver. Thus, it is essential to simplify or to facilitate handling of the dialysis system in order to limit the risks of wrong preparation or actions or the risk of contaminations of sterile fluidic elements from of the system.

Furthermore, a large amount of a dialysis solution is consumed to dialyze the patient during a single treatment for example about 120 liters for hemodialysis therapy.

Because of the large volume of dialysate solution to be managed, one solution is to recycle or regenerate the dialysate ongoing during treatment, for example once the dialysate solution has been used. This solution allows to use only a few liters (about 51 for hemodialysis therapy). To regenerate the dialysate, the system may comprise a sorbent device.

### SUMMARY

The present document discloses several features of a sorbent device. Each feature may be claimed in an independent claim.

A first aspect of the disclosure relates to a sorbent cartridge which may comprise a compressible layer (such as but not limited to a foam) in the sorbent column. This configuration may allow to:
overcome manufacturing challenges due to sorbent density variations resulting in imperfect cartridge packing,
avoid formation of cavities and inhomogeneities in sorbent column during transport, and
allow flexibility of using different sorbent configurations in one and the same casing (e.g., foam fills void space and thereby ensures perfect packing).

In one possible embodiment, the sorbent cartridge comprises:
A cartridge body,
A lid,
a first port (which may be optionally arranged on the cartridge body),
a second port (which may be optionally arranged on the lid),
An internal compartment through which the dialysate solution flows from the second port to the first port,
A sorbent column having at least one layer of cleaning material stored in the internal compartment, and
A compressible layer in an at least partially compressed state.

The cartridge may be constructed and arranged so that the dialysate solution entering the cartridge comes into contact with the compressible layer before coming into contact with the sorbent column (or other layer (such as the urease layer and/or the adsorption layer)). Even if the compressible layer may be arranged anywhere in the sorbent cartridge, it may be preferable to arrange the compressible layer at an end of the cartridge (in particular at an end of the internal compartment (e.g., at a top end of the internal compartment and/or at a bottom end of the internal compartment)).

The compressible layer may comprise at least one of a flexible behavior and an elastic behavior.

The compressible layer may be configured to act as a spring on the sorbent column.

The sorbent column may comprise a set of particles arranged in the cartridge body and held by the compressible layer. The compressible layer may exert a force on the sorbent column so as to prevent any free movement of the particles or to maintain the integrity of the sorbent column.

The compressible layer may be compressed to about 10-90%, or about 50%.

The compressible layer may comprise an open-cell sponge-type material allowing the passage of the dialysate solution to pass through it. The compressible layer may be configured to disperse/distribute/share the liquid evenly over the entire surface of the sorbent column.

The sorbent cartridge may further comprise a filter arranged between the compressible layer and the sorbent column or between the lid and the compressible layer.

The compressible layer may be compressed so as to compensate density fluctuations of the sorbent column or a volume change of the sorbent column.

The compressible layer may be arranged in the internal compartment against the lid upstream to the sorbent column. The pore size of the compressible layer may be comprised between 30 and 100 ppi (pores per inch), between 40 and 90 ppi, between 40 and 50 ppi, or between 70 and 90 ppi. The compressible layer may comprise biocompatible material.

The cartridge body may comprise a substantial conical shape and the compressible layer is arranged at a wider part of the cartridge body.

A second aspect of the disclosure relates to a sorbent device which may comprise an inner wall having a specific shape configured to enhance the evenness of the dialysate flow through the sorbent device. This configuration may allow to:
avoid dialysate sorbent by-pass at cartridge walls
combination of smaller steps and larger steps provides an even effect throughout the cartridge and facilitates cartridge manufacturing (molding)

In one possible embodiment, the sorbent cartridge comprises:
A cartridge body having an internal compartment, a first end, a second end, a first port (which may be optionally arranged on the first end), and a second port (which may be optionally arranged on the second end), and
A sorbent column having at least one layer of cleaning material stored in the internal compartment,

The cartridge body may further comprise inner wall extending from the first end to the second end. The inner wall may comprise a first series of stairs and a second series of stairs arranged on at least one step of the first series of stairs so as to control the flow of the dialysate solution in the vicinity of the inner wall.

The inner wall may be configured to generate turbulence in the vicinity of the inner wall so that the dialysate solution flows through the sorbent column substantially uniformly.

The step of the first series of stairs may be larger than the step of the second series of stairs. At least one of the first series of stairs and the second series of stairs may be configured in such a manner that the second end having an average diameter greater than the first end.

The first series of stairs may comprise at least two steps and/or the second series of stairs may comprise at least two steps. Each step of the first series of stairs may comprise the second series of stairs. The first series of stairs may comprise evenly spaced steps and/or the second series of stairs may comprise evenly spaced steps.

A third aspect of the disclosure relates to the composition of the sorbent device or a system using such a sorbent device.

For instance, the sorbent device may comprise a layer having a mixture of urease with activated carbon. This layer may not comprise any zirconium phosphate or zirconium oxide. This configuration may allow to:
increase stability and shelf-life of urease through unexpected stabilizing effect of activated carbon
facilitate cartridge assembly process by using urease directly in the form it is being manufactured

For instance, the sorbent device may comprise a homogeneous mixture of at least one of a ZP (zirconium phosphate), H₂O (Hydrous zirconium oxide), AC (Activated carbon), and a Na source, but no urease. This configuration may allow to:
avoid wastage of urease in layers where urease activity is not required
avoid urease leaching from upper layers in sorbent (increase biocompatibility)
eliminate risk of premature ammonia leaching in cartridge with insufficient urease activity (mitigation against effect from incorrect storage or incorrect use) (The problem may occurs in cartridges where urease is part of the (homogenous) absorber layer (the second layer))

For instance, a system may comprise at least one of a dialysate loop line, a sorbent device, and a supply line configured to add bicarbonate-equivalent salts (such as lactates or acetates) in the dialysate loop line. This configuration may allow to:
increase release of bicarbonate-equivalents to patient
compensate condition of acidosis in patient

For instance, a system may comprise at least one of a dialysate loop line, a sorbent device, and a supply line configured to add Na-salt (which may comprise at least one of chloride, lactate or acetate) in the dialysate loop line. This configuration may allow to:
prevent excessive removal of Na from patient (eg with extremely low urea concentrations)

In one embodiment, the sorbent cartridge comprises a urease layer comprising a mixture of urease and activated carbon and an adsorption layer comprising zirconium-based ion exchange particles. The components of the adsorption layer interact with the fluid either by adsorption or absorption.

The cartridge may be configured such that spent dialysate contacts the urease layer before the spent dialysate contacts the adsorption layer.

The urease layer may not comprise any ion exchange particles (e.g., group IV transition metal-based ion exchangers), or any cation exchange particles or any anion exchange particles. The adsorption layer may not comprise urease. The adsorption layer may comprise activated carbon. In one embodiment, the urease layer does not comprise any cation exchanger.

The zirconium-based ion exchange particles may comprise at least one of a cation exchange particles and anion exchange particles. The zirconium-based ion exchange particles may comprise zirconium phosphate, acid zirconium phosphate, sodium zirconium phosphate, zirconium oxide, zirconium hydroxide, zirconium oxide hydroxide, zirconium hydrous oxide, hydrous zirconium oxide, zirconium oxide with hydration, zirconium oxide without hydration, or combinations thereof. The zirconium-based ion exchange particles may comprise a homogenous mixture of zirconium phosphate and hydrous zirconium oxide.

The adsorption layer may further comprise sodium source. The sodium source may comprise sodium-carbonate and/or sodium-bicarbonate.

The sorbent cartridge may further comprise an inlet port and an outlet port. The urease layer may be arranged in the vicinity of the inlet while the adsorption layer is arranged in the vicinity of the outlet.

The urease of the urease layer may be immobilized.

In one possible embodiment, the apparatus for carrying out a dialysis treatment comprises a sorbent cartridge, and a dialysate circuit in fluid communication with the sorbent cartridge. The dialysate circuit may be configured so that the spent dialysate passes through the sorbent cartridge to clean the spent dialysate. The sorbent cartridge may comprise a urease layer comprising a mixture of urease and activated carbon and an adsorption layer comprising zirconium-based ion exchange particles.

The cartridge may be configured such that spent dialysate contacts the urease layer before the spent dialysate contacts the adsorption layer.

The urease layer may not comprise any ion exchange particles (e.g., group IV transition metal-based ion exchangers), or any cation exchange particles or any anion exchange particles. The adsorption layer may not comprise urease. The adsorption layer may comprise activated carbon. In one embodiment, the urease layer does not comprise any cation exchanger.

The urease layer may further comprise glucose. The adsorption layer may further comprise glucose. This may be used to act as an osmotic agent to control the dialysate osmolality during priming. The advantage may be that this allows to use priming dialysate which does not contain glucose. Therefore, the priming solution of the dialysate circuit may comprise a dialysate solution without glucose, or the dialysate solution may be generated during the priming process as described in the PCT/IB2023/060957 filed on Oct. 31, 2023 in the name of Nextkidney, the entire disclosure of which is incorporated herein by reference.

The zirconium-based ion exchange particles may comprise at least one of a cation exchange particles and anion exchange particles. The zirconium-based ion exchange particles may comprise zirconium phosphate, acid zirconium phosphate, sodium zirconium phosphate, zirconium oxide, zirconium hydroxide, zirconium oxide hydroxide, zirconium hydrous oxide, hydrous zirconium oxide, zirconium oxide with hydration, zirconium oxide without hydration, or combinations thereof. The zirconium-based ion exchange particles may comprise a homogenous mixture of zirconium phosphate and hydrous zirconium oxide.

The adsorption layer may further comprise sodium source. The sodium source may comprise at least one of sodium-carbonate, sodium-bicarbonate, and sodium hydroxide.

The sorbent cartridge may comprise an inlet port and an outlet port, the urease layer may be arranged in the vicinity of the inlet while the adsorption layer may be arranged in the vicinity of the outlet.

The urease of the urease layer may be immobilized.

The apparatus may further comprise a dialyzer in fluid communication with the sorbent cartridge, and the dialysate circuit is configured so that the spent dialysate passes from the dialyzer to the sorbent cartridge. The dialysate circuit may comprise a loop circuit comprising the dialyzer and the sorbent cartridge.

The present application claims the benefit of the priority of EP22205023.9, EP22205027.0, EP22205030.4, EP22205036.1, EP22205040.3, and EP22205043.7 filed on Nov. 2, 2022, and the priority of the PCT/IB2023/060957 filed on Oct. 31, 2023 in the name of Nextkidney, the entire disclosures of which are incorporated herein by reference.

### BRIEF DESCRIPTION OF DRAWINGS

The present disclosure will be better understood in light of the following detailed description which contains non-limiting examples illustrated by the following figures.

FIG. 1 illustrates a potential architecture of the system.

FIG. 2 illustrates a normal operating of the dialysate circuit during the treatment.

FIG. 3 illustrates an embodiment of a sorbent device.

FIG. 4 discloses a cross-sectional view of an embodiment of a sorbent device.

FIGS. 5a and 5b illustrate the general flow pattern of dialysate solution through a cavity of a sorbent device.

FIGS. 6a, 6b, 6c, and 6d illustrates a cross-sectional view of an embodiment of a sorbent device.

FIG. 7 discloses a cross-sectional view of an embodiment of a sorbent device.

FIGS. 8a, 8b, 8c, 8d; and 8e show the performance of different configurations of sorbent in a sorbent device.

FIG. 9 compares the urease activity of a urease layer comprising urease and activated carbon and a urease layer comprising urease but not activated carbon.

FIG. 10 discloses an exploded view of an embodiment of a sorbent device.

FIGS. 11a and 11b discloses a cross-sectional view of an embodiment of a sorbent device.

### LIST OF ELEMENTS

1 Fluid circuit of the system
2 Blood circuit
3 Dialysate circuit
4 First filter (e.g. dialyzer)
5 Blood cartridge
6 Dialysate cartridge
10 First bag (e.g. weighing bag)
11 second bag (e.g. additive bag)
12 Second filter (e.g. sorbent device)
13 First pump
14 Second pump
15 Third pump
16 Fourth pump
17 Valve or clamp
18 First sensor (e.g. pressure sensor)
19 Second sensor (e.g. air sensor)
20 Third sensor (e.g. level sensor)
21 Fourth sensor (e.g. temperature sensor)
22 Fifth sensor (e.g. ammoniac sensor)
23 Air flow device
24 Connector
25 Arterial line
26 Venous line
27 Drip chamber
29 Dialysate loop line
31 Supply line
32 Patient
100 System
101 Reusable part
102 Disposable part
103 Sensor
104 Sensing area
105 Actuator (e.g. valve actuator, pumping device, . . . )
106 Actuation area (e.g. valve, pumping head, . . . )
107 User interface device
108 Patient
109 Processor
200 Sorbent device
201 Body
202 Cavity
203 Inner wall
204 Lid
205 Inlet
206 Outlet
207 From the dialyzer/peritoneal cavity
208 To the dialyzer/peritoneal cavity
209 Foam
210 Chemical component
211 First layer
212 Second layer
213 First tube
214 Second tube
215 First connector
216 Second tube
217 Filter
218 First series of stairs
219 Second series of stairs

### DETAILED DESCRIPTION

The following detailed description includes references to the accompanying drawings, which form a part of the detailed description. The drawings show, by way of illustration, specific embodiments in which the disclosure may be practiced. These embodiments, which are also referred to herein as “examples,” are described in enough detail to enable those skilled in the art to practice the disclosure. The embodiments may be combined, other embodiments may be utilized, or structural, logical, and electrical changes may be made without departing from the scope of the present disclosure. The following detailed description is, therefore, not to be taken in a limiting sense, and the scope of the present disclosure is defined by the appended claims and their equivalents.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the claims, the singular forms “a”, “an”, and “the” encompass embodiments having plural referents, unless the content clearly dictates otherwise.

As used in this specification and the claims, any direction referred to herein, such as “top”, “bottom”, “left”, “right”, “upper”, “lower”, and other directions or orientations are described herein for clarity in reference to the figures and are not intended to be limiting of an actual device or system unless the content clearly dictates otherwise. Devices and systems described herein may be used in several directions and orientations.

As used in this specification and the claims, “have”, “having”, “include”, “including”, “comprise”, “comprising” or the like are used in their open-ended sense, and generally mean “including, but not limited to”.

As used in this specification and the claims, the term “or” is generally employed in its sense including “and/or” unless the content clearly dictates otherwise.

As used in this specification and the claims, “at least one of A, B, and C”, “at least one of A, B or C”, “selected from the group consisting of A, B, C, and combinations thereof” or the like are used in their open ended sense including “only A, or only B, or only C, or any combination of A, B and C” unless the content clearly dictates otherwise.

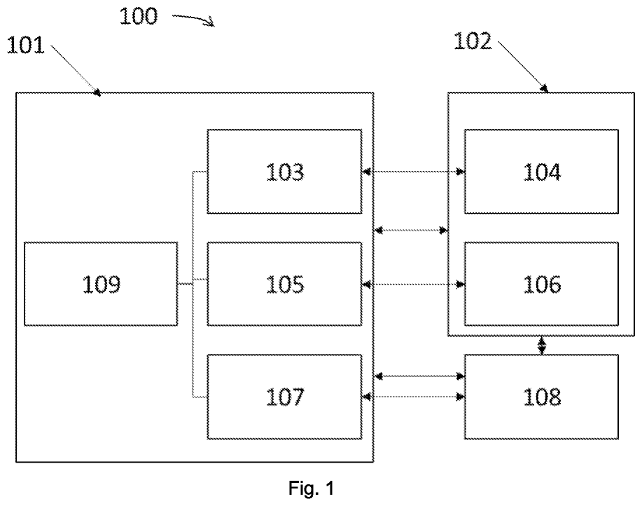

According to one embodiment of the disclosure as shown by FIG. 1, the system (100) may comprise a reusable part (101) and a disposable part (102). The disposable part (102) may comprise the elements which have to be discarded after a predetermined number of uses, for example, after a single treatment. The working life of the disposable part (102) may directly depend on the number of treatments. These elements may be the elements which have been wetted by the medical fluid (such as dialysate) or by the patient fluid (such as blood).

The reusable part (101) may comprise the expensive elements for example the sensor (103), the electronic part, the user interface device (107), the actuator (105) of the valve or of the pump, the processor (109) or the memory. The reusable part (101) is successively used with several disposable parts (102). The reusable part (101) may comprise components which may be replaced when the components are too worn, become broken or after a predetermined period of time, but much longer than a single treatment. The change of the reusable part may depend on the component wear.

The Reusable part (101) may be configured to be operatively coupled to the disposable part (102). A sensor (103) may be configured to be operatively coupled to a sensing area (104) of the disposable part. An actuator (105) may be configured to be operatively coupled to an actuation area (106) of the disposable part (102). The reusable part (101) (e.g. the user interface device (107)) may be configured to provide information to the patient (108) and/or to receive instruction from the patient (108). At least one of the sensor (103, the actuator (105) and the user interface device (107) may be connected to a processor (109). The disposable part (102) may be connected to or in contact with the patient (108) at least during the treatment.

The following description discloses a hemodialysis treatment system using a sorbent device, but a similar sorbent device may be used with a peritoneal dialysis treatment. The type of treatment cannot be understood as a limitation of the use of the sorbent device disclosed in the present document.

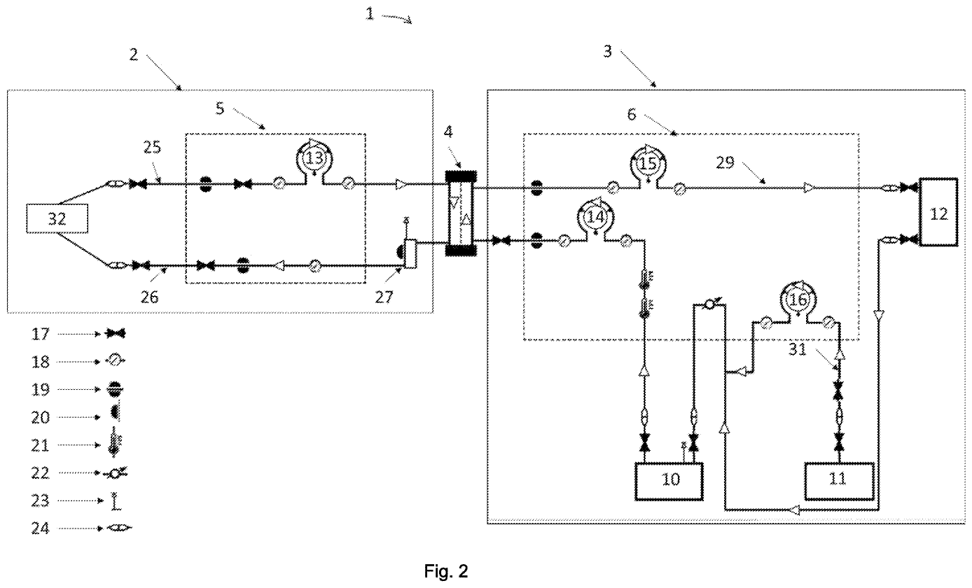

According to one embodiment as shown by FIG. 2, the system may comprise a fluid circuit (1). The fluid circuit of the system (1) may comprise a blood circuit (2), a dialysate circuit (3), and a first filter (e.g. dialyzer) (4). The elements of the fluid circuit wetted by a fluid may be a part of the disposable part (e.g. as disclosed above). The system may comprise at least one of a first sensor (18), a second sensor (19), a third sensor (20), a fourth sensor (21), a fifth sensor (22), and other sensors. These sensors may be a part of the reusable part (e.g. as disclosed above).

The blood circuit (2) may comprise at least one of a connector (24), an arterial line (25), a venous line (26), and a first pump (13). The arterial line (25) and/or the venous line (26) may be intended to be connected to the patient (32) via for example a catheter (not shown here), for example during the treatment. The first pump may be configured to move a fluid (for example blood of the patient) from the arterial line to the venous line in a normal operation (for example during the treatment) and from the venous line to the arterial line in a reversed operation (for example during at least a part of the priming). The first pump may be controlled by the processor.

The blood circuit (2) may further comprise at least one of a drip chamber (27), a pressure sensor (18), an air sensor (19), a level sensor (20), and a valve or clamp (17). The drip chamber (27) may comprise an air flow device (23). At least some of the above elements may be arranged in a blood cartridge (5).

The dialysate circuit (3) may comprise at least one of a dialysate loop line (29), a second pump (14), a third pump (15), a second filter (12) (for example a sorbent device), and a first bag (10) (also called weighing bag). The dialysate circuit (3) may comprise at least one of a supply line (31) connected to a second bag (11). The supply line may comprise a fourth pump (16). The dialysate circuit may further comprise at least one of a pressure sensor (18), a temperature sensor (21), an ammonia sensor (22), a connector (24), and a valve or clamp (17). At least some of the above elements may be arranged in a dialysate cartridge (6).

The blood circuit (2) and the dialysate circuit (3) may be fluidically connected to the first filter (4) for example a dialyzer. The first filter may comprise a blood compartment connected to the blood circuit and a dialysate compartment connected to the dialysate circuit. Both may be separated by a permeable membrane.

The weighing bag (10) may be arranged on a warmer and/or a weighing scale (not shown). The weighing scale may be connected to a processor to determine the volume (or the weight or a data related to the weight or volume) of fluid (liquid) stored in the weighing bag (10).

In one embodiment, the first bag may be configured to store a dialysate solution required for the treatment. The second pump (14) moves the solution from the first bag to the first filter. By passing through the first filter the dialysate solution is deemed spent and cannot be used a second times. Afterwards, the third pump (15) moves the spent dialysate solution to the second filter (12) in order to “clean” the spent dialysate solution (e.g., to remove toxins such as urea). Then the cleaned dialysate solution is moved back to the first bag.

In some embodiments, the sorbent removes (from the dialysate solution) also some electrolyte (or essential ions) required to the treatment which subsequently have to be re-added or re-constituted. Therefore, during the treatment, the second pump (14) and the third pump (15) may be operated to move the dialysate through the dialysate loop line and the fourth pump (16) may be also operated to inject additive solution into the dialysate loop line (29). The additive solution may be added to the dialysate loop line (29) between the second filter (12) and the first bag (10) for example upstream of the first bag (10) and/or downstream of the second filter (12). It may also be added between the first bag and the first filter, for example downstream of the first bag and upstream of the first filter.

The second bag (11) may initially store the additive solution which may comprise electrolyte (or essential ions) required to reconstitute the dialysate solution. The second bag may be configured to store a volume of fluid comprised between 0.5 L and 5 L, preferentially between 1 L and 4 L, for example 2.5 L. The volume of this bag may depend on the time duration of the treatment and on the concentration of the electrolyte. The additive solution may comprise water and electrolyte (such as for example at least one of magnesium, calcium, and potassium).

In one embodiment, the sorbent (12) may comprise at least one of active carbon, ion exchangers (such as zirconium phosphate and/or hydrous zirconium oxide), and one or more enzyme (e.g. urease). During the treatment as explained above, the sorbent may be configured to remove toxins (such as urea and other). The toxin removal may involve at least one of the following phenomena: (i) adsorption (or absorption), (ii) catalysis, and (iii) ion exchange, at the component(s) comprised in the sorbent. As a result of the toxin removal, gas may be generated (such as CO2).

Therefore, in some embodiments, the first bag may be configured to store the dialysate solution and to collect at least one of the cleaned dialysate, additive solution, and gas.

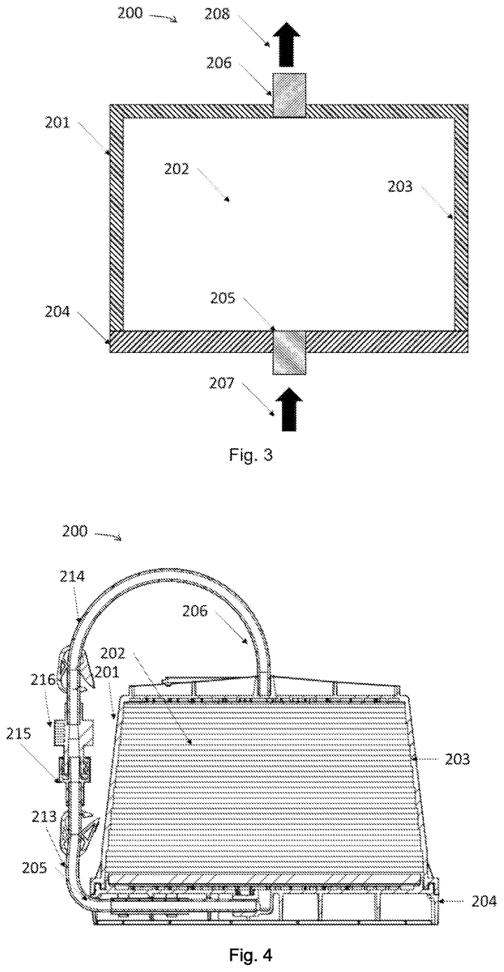

In one embodiment as illustrated by FIG. 3, a sorbent cartridge (200) (also called sorbent or sorbent device) may comprise at least one of a body (201), a lid (204), an inlet port (205), and an outlet port (207). The body may comprise a cavity (202) configured to store at least one of a chemical component used to clean a dialysate solution. The cavity may be defined by at least one inner wall (203) and may be closed by the lid (204). The inlet port and the outlet port are configured to provide a fluidic communication to the cavity (also called internal compartment). The inlet port may be configured to allow the supply of dialysate (for example spent dialysate) to the sorbent and the outlet port may be configured to allow the discharge of the dialysate (for example cleaned dialysate).

A sorbent cartridge may include multiple layers that comprise a similar or substantially chemical composition in each given layer. Flow distribution in a given cartridge layer of the sorbent cartridge can vary across the layer. Channeling phenomenon can occur in a peripheral region of a cartridge layer or layers of a cartridge that are located nearer to the cartridge wall. Fluid flow can increase in the peripheral region of a layer or layers at the expense of a central region thereof located further from the cartridge wall. This is undesirable as it can result in separate regions of overly-used material and unused (or underused) material in the same layer of the cartridge. This can lead to inefficient treatment performance, early or premature exhaustion of a cartridge component, shortening of the useful life of cartridge, unused material in the spent cartridge, or combinations of these problems. Sorbent cartridge designs would be preferred that can further reduce or prevent variations in flow distribution from occurring in the sorbent cartridge.

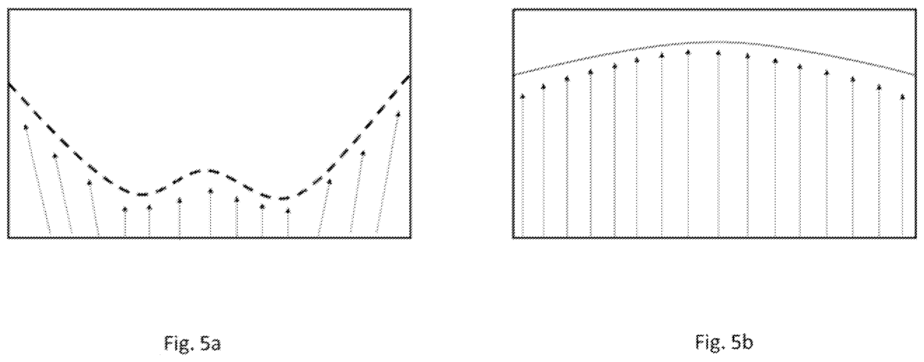

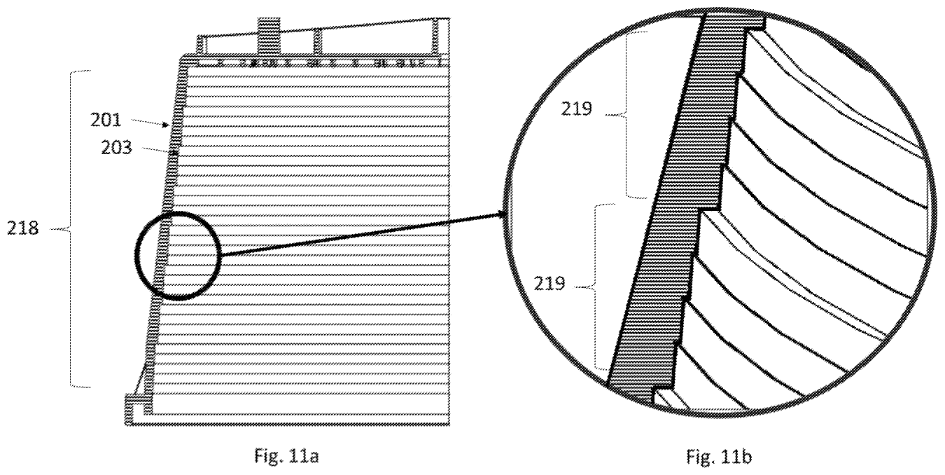

FIGS. 4, 11a, and 11b disclose a cross sectional view of a sorbent device (200) comprising a conically shaped body which is of conical cylindrical shape (truncated cone). In this example, the body (201) may comprise an inner wall (203) subdivided by a first series of stairs (218) and a second series of stairs (219).

The first series of stairs may comprise at least one step comprising the second series of stairs. FIG. 11a shows a cartridge body comprising a first series of stairs wherein each step of the first series of stairs comprises a second series of stairs. In this example, the step height of the first set of stairs is greater than the step height of the second set of stairs and the depth of the steps in the first set of stairs is greater than the height of the steps in the second set of stairs. Nevertheless, the depth and height of each step can vary from step to step and/or series to series and can be configured to achieve the desired objective. FIG. 11b shows a zoom of a first series of stairs (218) comprising a second series of stairs (219).

The size of the stairs of the first series of stairs may be comprised between 0.5 and 2 mm (e.g., 1 mm). The size of the stairs of the second series of stairs may be comprised between 10 and 500 μm (e.g., 100 μm). One reason for these sizes may be that the small stairs are in the same order of magnitude as the particle size in the adsorber material.

For instance, the first series of stairs may comprise 7 evenly spaced larger steps, wherein each of the subdivided sections further may comprise a second series of stairs comprising 3 evenly spaced smaller steps. The inner wall can be subdivided by steps of uniform or varying step size. Each section subdivided by the smaller steps may have a draft angle for easy moldability. The combined arrangement of all steps may define the overall cone angle.

When in use, dialysate may enter the conical cartridge body through the side of the large radius and may leave through the smaller radius side.

The cone radii and height may be chosen such that the resulting volume is larger or equal to the volume of the sorbent filling (the sorbent column), and such that, when in use, a pressure drop caused by the flow resistance of dialysate passing through the sorbent column remains below a desired maximum pressure drop (e.g. 0.6 bar at 300 mL/min). The size of the small steps may be chosen to be in the range of the average size of sorbent particles (e.g. 50-150 μm), thereby preventing sorbent bypass along the cartridge inner walls. The presence of the larger steps induces areas of turbulent flow and thereby further prevents sorbent bypass and contributes to even out flow inconsistencies.

In one embodiment, the section between the base of the cone (largest radius) and the first large step may comprise a total of 5 small steps (for example), thereby providing for additional volume used as excess volume. This facilitates the sorbent filling process by providing extra volume.

In one embodiment, the sorbent cartridge comprises at least one of a first layer and a second layer comprising components configured to clean spent dialysate. At least one layer may comprise a homogenous mixture of two components and/or at least one layer may be arranged in a part of the cavity comprising at least two large steps and/or at least two small steps. For instance, a homogenous layer may be arranged in the cavity and extending sufficiently to fill an area (of the inner wall) comprising at least two steps of the second series of stairs and/or at least two steps of the first series of stairs.

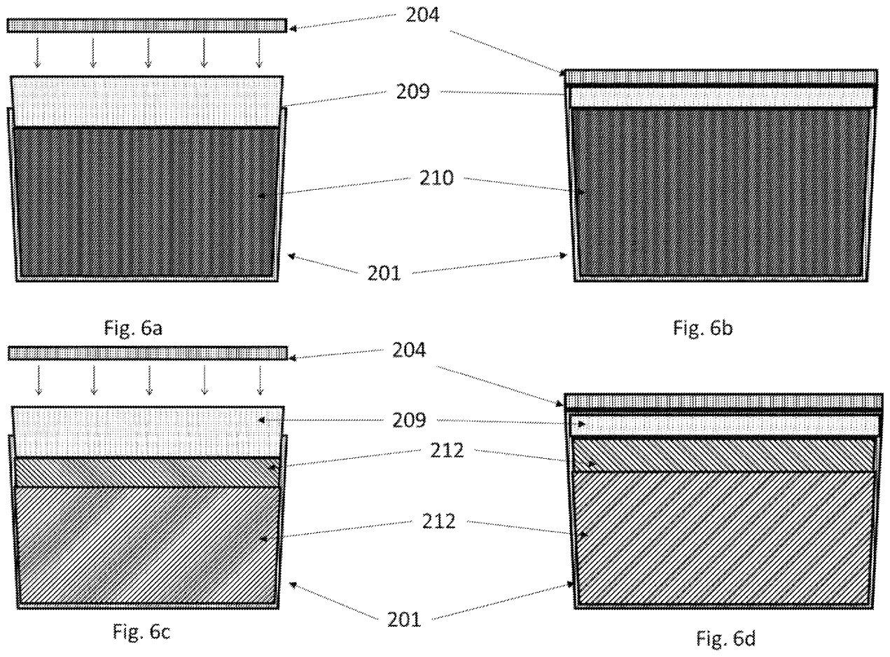

FIGS. 5a and 5b show the difference of the fluid flow pattern between a sorbent device having smooth inner walls (FIG. 5a) and a sorbent device having optimized shape inner wall (for example as disclosed above). In absence of steps (FIG. 5a), the fluid flow along the inner wall is faster than that in the body of the sorbent column. This results in a more rapid advance of the fluid front, and subsequently of the sorbent exhaustion front, in the vicinity of the walls, relative to the body of the sorbent column. The sorbent column will thereby exhaust more rapidly at the areas along the inner wall, where toxin breakthrough will happen prematurely, before most of the sorbent in the body of the sorbent column has been used up. The unused sorbent is wasted and the size of such a sorbent device has to be increased, in order to provide the desired amount of toxin removal before toxin breakthrough. In presence of steps, the flow along the inner wall is slowed down until it is even with the flow in the body of the sorbent column. Like that, toxin breakthrough happens at a much later time, when much more of the sorbent in the inner areas of the sorbent column has been used.

In one embodiment, the body may comprise plastic parts. A draft angle of the straight sections in between the steps at the side walls may be chosen to facilitate the molding process by allowing for easy tool release. The body may comprise ABS, PC, PP, or any other suitable polymer material.

In one embodiment, during assembly, the body may be placed such that the larger radius, open side of the cone is facing upwards. Sorbent materials may be filled from the top until a desired fill level. Afterwards, the lid may be placed on the body. The lid (204) may be fixed to the cartridge body, e.g. by gluing or welding.

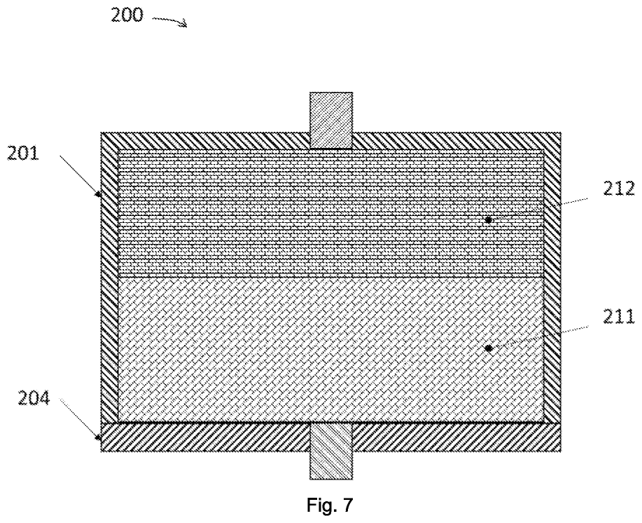

FIGS. 6a and 6b illustrate a cross sectional view of an embodiment of a sorbent device (200). In this embodiment, the sorbent device (200) may further comprise a foam (209). The foam may have flexible and/or elastic behavior. FIG. 6a shows a foam in expanded form while FIG. 6b shows the same foam but compressed. During assembly, sorbent materials (components configured to clean spent dialysate) may be filled from the top until a desired fill level. The remaining free volume of the body is then filled with foam (209), which may protrude above the top surface of the body (201). The protruding foam may then be pressed down with for example the lid (204), thereby compressing the foam (209). The lid may be fixed to the cartridge body, e.g. by gluing or welding. Thanks to the elasticity of the foam, a spring force may act onto the sorbent filling, thereby compressing it and preventing the formation of loosely packed areas or cavities.

The foam may be cut to cylindrical shape (disk) of 10-40 mm height (for example), and a radius equal or larger than the larger radius of the conical body. For example, the radius of the cylindrical disk may be 1 mm larger than the large radius of the body, and the height may be 20 mm. During assembly, the foam may be compressed to about 10-90% or 20-80% of its uncompressed height. For example, it may be compressed to about 50% of its uncompressed height.

The foam may comprise an open-cell sponge-type material, which readily allows for the passage of liquid. The foam may comprise a biocompatible polyurethane material.

In one embodiment, the force acting by the compressed foam onto the sorbent material (spring force) may prevent any free movement of the sorbent particles (for example due to vibration or other agitation during transport) and thereby maintains the integrity of the sorbent column (sorbent packing). The foam may be positioned at the larger radius side of the cartridge body (casing), e.g. at the dialysate inlet side.

Alternatively to using one single disk of elastic foam, 2 or more layers may be combined to obtain a total desired height of foam, e.g. 2 layers of 10 mm foam may be combined to a total foam height of 20 mm.

In one embodiment, the foam may be separated from the sorbent column by a filter (for example filter paper) or may be in direct contact with the sorbent column (chemical components).

The foam layer may be configured to compensate density fluctuations of the chemical components, resulting in fluctuations of the chemical components height within the cavity.

These fluctuations may result from normal expected fluctuations of the chemical components (e.g. introduced during the sorbent manufacturing process).

The foam layer may be configured to compensate volume changes of the sorbent column arising from transport or storage, such as tighter packing due to vibration during transport.

The foam layer may be configured to compensate or to be used as an adjustable, low-cost filler material to allow for flexibility of the sorbent material fill level, where desired. This allows the use of the same body for smaller, more economical cartridges, where desired.

In one embodiment, the position of fluid inlet and/or outlet may be central relative to the body of the sorbent. The inlet and/or outlet may comprise a tube which may be glued to at least one of the body and the lid.

In one embodiment disclosed on FIG. 4, the inlet tube may be arranged at least partially along and/or through the lid. The lid may comprise a recess through which the inlet tube may be arranged such that the inlet tube cannot be kinked or otherwise affected by the weight of the sorbent device. The width of the recess may be larger than the inlet tube. The inlet tube may be fastened to the lid by pinching, welding and/or gluing.

In one embodiment, the sorbent may comprise a flow distribution means having a shape of spider-web at inlet and outlet. The foam may be in direct contact with the spider web or there may be a filter paper in between spider web and foam. In one embodiment, the foam may be configured to dispense the flow in place of the flow dispensing means, or contribute to dispensing the flow with the flow dispensing means.

In one embodiment, the lid may comprise transparent material in such a way to allow UV gluing.

In one embodiment, the body and/or the lid may comprise a skirting, step/shoulder to allow for ultrasonic welding, and/or a structure of stabilizing ribs. The body may further comprise a structure of ribs to stabilize the step or shoulder.

In one embodiment, the sorbent may comprise a handle to transport the sorbent. The body and/or the lid may comprise a fastening means to fasten the handle to the sorbent. The fastening means may comprise at least one of a clip and mushroom head.

In one embodiment, the inlet may comprise a first tube (213) and a first connector (215) intended to be connected to the dialysate loop line at least during the treatment (for example downstream the dialyzer). The outlet may comprise a second tube (214) and a second connector (216) intended to be connected to the dialysate loop line at least during the treatment (for example upstream the dialyzer). During storage or transport, the first connector may be configured to be connectable to the second connector.

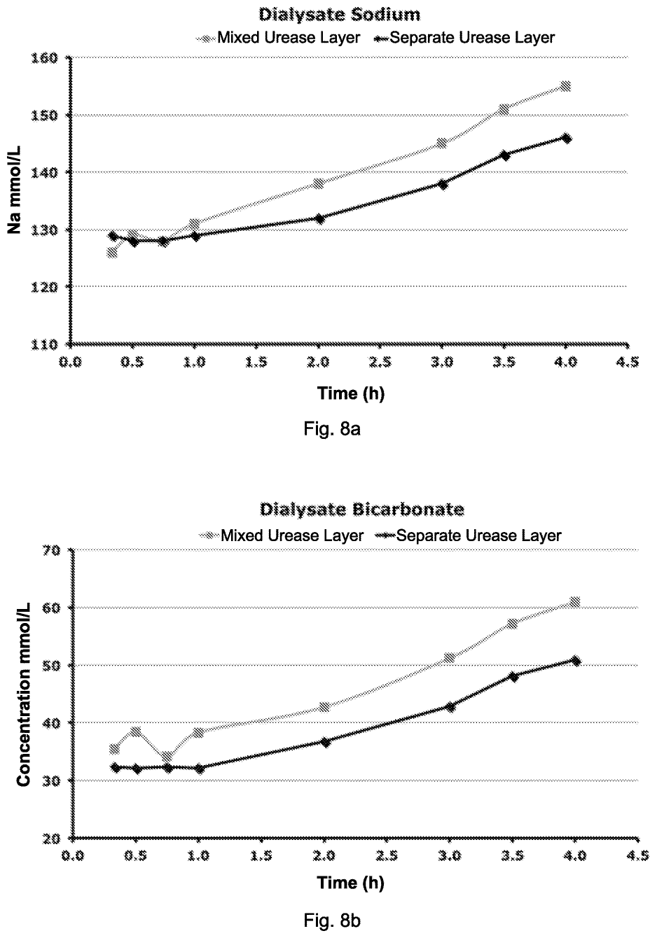

In one embodiment as shown by FIG. 7, the sorbent may comprise at least one of a first layer (211) of chemical component and a second layer (212) of chemical component. The first layer may be a urease layer and the second layer may be an adsorption layer.

The first layer may comprise at least one of urease and activated carbon. The first layer may comprise urease provided as intermixture with activated carbon particles. This may be obtained by blending urease with activated carbon particles. The activated carbon particles may be obtained from any common source, e.g. coconut shells. The urease may be in the form of urease particles. The urease particles may be immobilized urease. The ratio of urease to activated carbon may, for example, be 1:1-1:100. The urease layer may comprise covalently immobilized urease, for example Immobilized on cellulose. The urease may be intermixed with activated carbon at a ratio of 1:8.

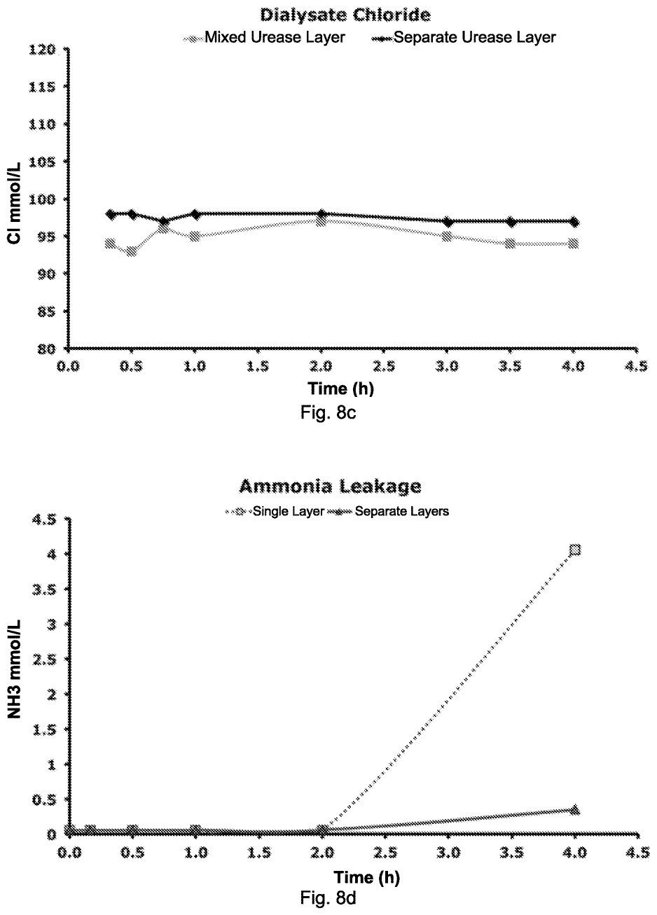

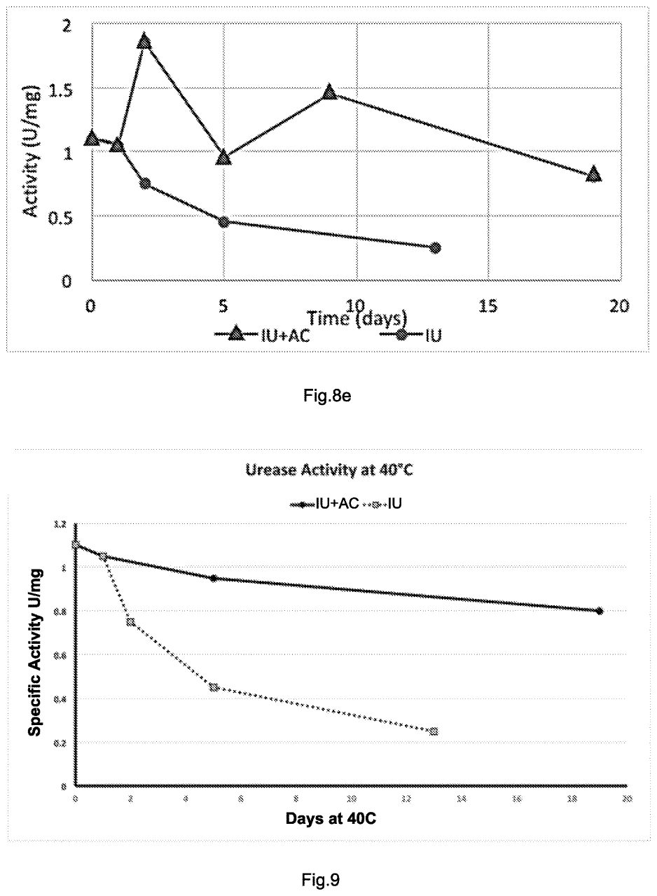

The intermixture of urease and activated carbon may provide at least one of following advantages:
a stabilizing effect for urease,
significantly improving shelf-life,
providing a matrix for urease, to dilute it to a concentration that can be handled during manufacturing, and
reducing activity losses during manufacturing, sterilization (where performed), transport, and storage (see for example FIGS. 8e and 9).

This stabilization effect may at least in parts be related to anti-oxidative properties of activated carbon. The activated carbon may provide stabilizing environment for urease. Furthermore, where the intermixture of AC and IU is already performed as part of the IU manufacturing process, the cartridge assembly process may be facilitated by providing the immobilized urease product as-is in a single layer (no further mixing and proportioning required). Therefore, the immobilized urease prevents urease leakage and mixed with activated carbon stabilizes enzyme.

Such a homogenous mixture is intrinsically equilibrated, reduces pressure drop and is shape independence.

In one embodiment, the first layer does not comprise ion exchange materials, or any cation exchange materials or any anion exchange materials, such as zirconium-based cation or anion exchanger particles. For example, the first layer does not comprise zirconium oxide, hydrous zirconium oxide, zirconium phosphate, or metal that forms an insoluble phosphate, . . . .

The first layer comprising a mixture of immobilized urease with activated carbon may be separated from another layer (for example the second layer) which may comprise ion exchange particles, e.g. zirconium-based ion exchange particles or other similar chemical elements. The first layer and the second layer may be separated by a filter (such as a filter membrane, such as a filter paper). Each layer may be separated by a filter.

In one embodiment the second layer may comprise a homogeneous mixture of ZP, HZO, optionally further comprising AC and/or a Na-source such as Na-carbonate or Na-bicarbonate. The second layer may not comprise urease.

The first layer may be arranged closer to the inlet, while the second layer may be arranged closer to the outlet. The first layer may be arranged adjacent to the second layer. The first layer may be arranged upstream of and/or close to the second layer. For example, the first layer may be arranged to be followed by the second layer.

Some of the advantages of the exclusion of urease from the second layer may be:
reduce cartridge manufacturing costs by avoiding urease wastage in a layer where urease function is neither required nor desired.
ensure that all urea hydrolysis and conversion to ammonia is completed in this first layer, before the dialysate reaches the second layer where ammonia will be bound.
prevent unwanted urea hydrolysis and ammonia formation in deeper layers (more downstream) of the sorbent column, which may result in early, slow exhaustion and unwanted early release of ammonia to the patient. This scenario may occur in the case of excessive amounts of urea from the patient, or unexpected loss of urease activity due to use errors such as incorrect storage or incorrect use. In those scenarios, the limitation of urease to a separate, preceding layer will only result in incomplete urea hydrolysis (low harm), rather than in premature release of ammonia (higher harm).
limit or eliminate the risk of urease leakage to the patient, thus significantly improving the biocompatibility of the sorbent.

As described above, the second layer may further comprise a sodium source (such as but not limited to Sodium bicarbonate). This addition may improve control of Na, HCO3, and PH.

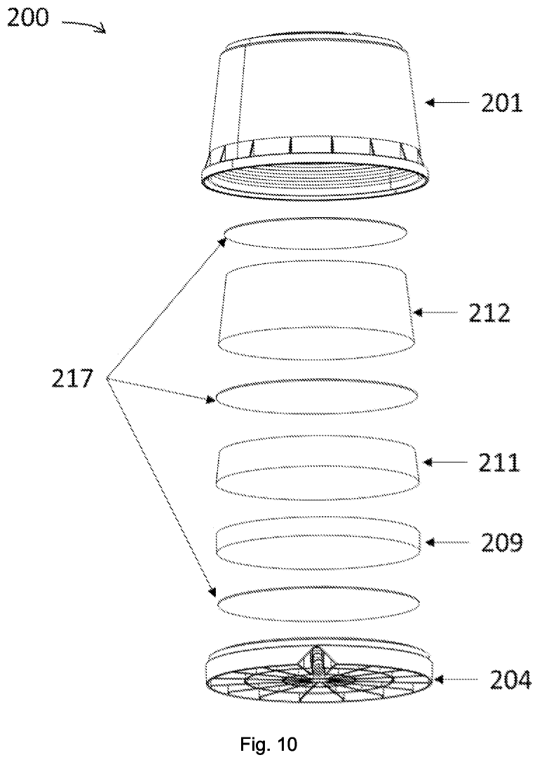

FIG. 10 show an example of a sorbent cartridge comprising cartridge body (201) having a cavity in which is stored:
a first layer (211) which may comprise a homogenous mixture of urease and activated carbon and
a second layer (212) which may comprise a homogenous mixture of ZP, HZO, AC and NaHCO₃.

The sorbent cartridge may further comprise a foam (209). FIG. 10 also shows filter (217) which may be arranged upstream to the first layer (and the foam), between the first layer and the second layer and/or downstream of the second layer.

FIGS. 8a, 8b, 8c, 8d, and 8e show the performance of different configurations of sorbent in a sorbent device. The dotted line in the figures shows the performance of a sorbent of one single, homogenous layer of a ZP-HZO-AC-urease intermixture, as described elsewhere. The solid line in the figures shows the performance of a sorbent according to the present invention, containing identical quantities of individual sorbent components, but separated into a first layer of a urease-AC intermixture without ion exchange particles, and a second layer of a ZP-HZO-AC intermixture without urease. As can be seen in FIG. 8a and FIG. 8b, the sorbent according to the present invention provides dialysate regeneration at better control of sodium and bicarbonate in regenerated dialysate. Starting at similar concentrations for sodium (FIG. 8a) and bicarbonate (FIG. 8b), the single-layer sorbent leads to a larger increase of the sodium and bicarbonate concentration in regenerated dialysate after 4 h and thereby to a larger change. The dialysate chloride concentration (FIG. 8c), on the other hand, remains approximately unchanged. FIG. 8d shows the effect on the extend of ammonia breakthrough. Although both sorbents contain the same quantity of urease and cation exchanger (ZP), the single-layer sorbent shows extensive ammonia breakthrough after 4 h, while the sorbent according to the present invention shows only minimal signs of exhaustion and ammonia breakthrough after 4 h. Hence, the 2-layer sorbent configuration is more efficient and more economical then the single-layer sorbent configuration.

According to an embodiment, the additive solution (for example stored in the second bag (11)) may comprise at least one of chloride salts (KCl), MgCl₂, CaCl2 and salts of a weak acid (such as lactate or acetate salts of Ca, Mg and/or K). The additive solution may comprise a mixture of at least one of Ca-lactate, Mg-lactate and K-chloride. Alternatively, the additive solution may comprise a mixture of at least one of Ca-lactate, Mg-lactate and K-lactate. We assume that the patient can metabolize lactate ions to bicarbonate ions.

One of the advantages of such an additive solution is that it may increase the amount of bicarbonate, rather than chloride in the patient, thereby countering the effects of metabolic acidosis commonly present in dialysis patients. Furthermore, the release of lactate may increase the buffer capacity of regenerated dialysate relative to chloride.

In one embodiment, the additive solution (for example stored in the second bag (11)) may further comprise a soluble Na-salt. This Na-salt may comprise at least one of NaCl, Na-lactate or Na-acetate. The Na-salt may comprise Na-lactate. The addition of Na-lactate to the cleaned dialysate may help to prevent excessively low plasma Na concentrations in the dialysate and in the patient. Furthermore, it may increase the buffer capacity of regenerated dialysate and counter the effects of metabolic acidosis.

1. A sorbent cartridge configured to clean dialysate solution, comprising:
a cartridge body,
a lid,
a second port,
a first port,
An internal compartment through which the dialysate solution flows from the second port to the first port,
A sorbent column having at least one layer of cleaning material stored in the internal compartment, and
A compressible layer in an at least partially compressed state.

2. The sorbent cartridge according to claim 1, wherein the cartridge is constructed and arranged so that the dialysate solution entering the cartridge comes into contact with the compressible layer before coming into contact with the sorbent column.

3. The sorbent cartridge according to claim 1, wherein the compressible layer comprises at least one of a flexible behavior and an elastic behavior.

4. The sorbent cartridge according to claim 1, wherein the compressible layer is configured to act as a spring on the sorbent column.

5. The sorbent cartridge according to claim 1, wherein the sorbent column comprises a set of particles arranged in the cartridge body and held by the compressible layer.

6. The sorbent cartridge according to claim 5, wherein the compressible layer exerts a force on the sorbent column so as to prevent any free movement of the particles or to maintain the integrity of the sorbent column.

7. The sorbent cartridge according to claim 1, wherein the compressible layer is compressed to about 10-90%, or about 50%.

8. The sorbent cartridge according to claim 1, wherein the compressible layer comprises an open-cell sponge-type material allowing the passage of the dialysate solution to pass through it.

9. The sorbent cartridge according to claim 1 further comprises a filter arranged between the compressible layer and the sorbent column or between the lid and the compressible layer.

10. The sorbent cartridge according to claim 1, wherein the compressible layer is compressed so as to compensate density fluctuations of the sorbent column or a volume change of the sorbent column.

11. The sorbent cartridge according to claim 1, wherein the compressible layer is arranged in the internal compartment against the lid upstream to the sorbent column.

12. The sorbent cartridge according to claim 1, wherein the compressible layer comprises biocompatible material.

13. The sorbent cartridge according to claim 1, wherein the pore size of the compressible layer is comprised between 30 and 100 ppi, between 40 and 90 ppi, between 40 and 50 ppi, or between 70 and 90 ppi.

14. The sorbent cartridge according to claim 1, wherein the cartridge body comprises a substantial conical shape and the compressible layer is arranged at a wider part of the cartridge body.

15. (canceled)

16. (canceled)

17. (canceled)
